(19) United States
(12) Patent Application Publication
(10) Pub. No.: US 2008/0275111 A1
(43) Pub. Date: Nov. 6, 2008

(54) Novel Use of Lignan Compounds

(76) Inventors: Jae-Kwan Hwang, Gyeonggi-do (KR); Jung-Soo Han, Busan (KR); Chol-Seung Lim, Daejeon (KR); Daqing Jin, Daejeon (KR); Sun-Hee Lee, Gyeonggi-do (KR); Kyu-Lee Han, Seoul (KR)

Correspondence Address: JHK LAW, P.O. BOX 1078, LA CANADA, CA, 91012-1078, US

(21) Appl. No.: 11/909,976

(22) PCT Filed: Mar. 31, 2006
(86) PCT No.: PCT/KR2006/001212

§ 371 Date: Sep. 27, 2007

(30) Foreign Application Priority Data
Mar. 31, 2005 (KR) 10-2005-0026963

(51) Int. Cl. A61K 31/36; A01N 31/14

(52) U.S. Cl. 514/464; 514/721

(57) ABSTRACT

The present invention relates to the novel use of lignan compounds represented by Formula I. More particularly, the present invention relates to a pharmaceutical composition for treating or preventing a brain disease, comprising a lignan compound represented by Formula I or a Myristica fragrans extract as an active ingredient, as well as the method and use for treating or preventing a brain disease using the lignan compound. The lignan compound represented by Formula I has the effects of antioxidation, brain cell protection and antiinflammation. Accordingly, said lignan compound will be highly useful for treating or preventing a brain disease.

### FIELD OF THE INVENTION

The present invention relates to the novel use of lignan compounds. More particularly, the present invention relates to a pharmaceutical composition for treatment or prevention of brain diseases, comprising lignan compounds or the extract of Myristica fragrans, a method for treatment or prevention of brain disease using the same, and use thereof.

### BACKGROUND OF THE INVENTION

As an increase of the human's life span and a progression to an aging society, brain diseases such as cerebral apoplexy, dementia and Parkinson's disease are increased. The brain diseases feature that death or degeneration of certain brain cells is progressed temporarily or for a long time. Because the dead brain cells are not restored, the dead of brain cell leads to mortal damage of brain function. Especially, the incompletion of brain function accompanying the progressive weakness of cognitive function, sensory function, movement function and whole body function results in changes of characteristics and behavior, thus patients will face the situation that they cannot control themselves. The main factors of the brain cell death include oxidative toxicity by oxidative stresses, excitatory toxicity and apoptosis, and each of them causes cell death through specific signal transduction pathway, respectively.

Particularly, in patients suffering from cerebral apoplexy, brain damage, Alzheimer type dementia and Parkinson's disease, it is suggested that a main factor of brain cell death is the oxidative damage of proteins, nucleic acids and lipids after accumulation of reactive oxygen species. Especially, the oxidative stress by free radicals has been reported to be a main factor of cell death occurred in each tissue of a body, and has also been suggested to be a main mechanism of cell death in brain diseases (Schapira, A. H., Curr. Opin. Neurol., 9(4):260-264, 1996). The evidences that the free radicals are associated with the death of neuronal cells in the brain disease includes the formation increase of reactive oxygen species after ischemia and inhibitory effects of ischemic neuronal cell death by antioxidants (Flamm, E. S. et al., Stroke 9(5): 445-447, 1978; Chan, P. H., J. Neurotrauma 9 Suppl. 2:S417-423, 1992), Fe²⁺ increase in the striatum of Huntington's disease (Dexter, E. T. et al., Ann. Neurol., 32 Suppl.:S94-100, 1992), the formation of free radicals by beta-amyloid shown in Alzheimer's disease (Richardson J. S. et al., Ann. N.Y. Acad. Sci., 777:362-367, 1996), point mutation in Cu/Zn SOD-1 gene in amyotrophic lateral sclerosis (ALS) (Rosen, D. R. et al., Nature, 362(6415):59-62, 1993), etc.

Additionally, glutamate, an excitatory neurotransmitter, functions as a neurotransmitter at a normal status. However, the glutamate causes the neuronal cell death when it is overexpressed due to various reasons. Overactivity of the glutamate receptors such as NMDA, AMPA and kainate receptors are also known as a main factor of neuronal cell death (Choi D. W. Neuron, 1:623-634, 1988). It was found that the neuronal toxicity by glutamate is associated with the neuronal cell death in ALS. This was supported that disorder of glutamate synthetase, disorder of glutamate transport proteins and increase of glutamate receptor proteins in ALS patients are found (Rothstein, J. D. Clin. Neurosci., 3(6):348-359, 1995; Shaw, P. J. et al., J. Neurol., 244:Suppl 2 S3-14, 1997).

Moreover, apoptosis as another factor of the brain cell death was reported. The apoptosis is a main type of the cell death shown in ischemia, brain damage, vertebra damage, Alzheimer type dementia and Parkinson's disease (Smale et al., Exp. Neurol., 133:225-230, 1995; Crow et al., Nat. Med., 3:73-76, 1997).

These reports show that the brain cell death by oxidative toxicity, excitatory toxicity and apoptosis are main factors of various brain diseases, and thus the development of drugs treating the brain diseases has focused on inhibition of oxidative toxicity and excitatory toxicity and/or inhibition of brain cell apoptosis.

Meanwhile, lignan refers to a group of natural compounds where n-phenylpropanes are linked by the β-site of n-propyl side chains and is widely distributed in nature. There have been studies on the various physiological activities of lignan, such as blood glucose-lowering, anticancer, anti-asthmatic and whitening activity. For example, it was reported that lignans isolated from sesame, such as sesamin, episesamin, sesaminol, sesamolin and episesaminol, have anti-inflammatory effects (Korean Patent Laid-Open Publication No. 1997-7001043), and lignan compounds isolated from Magnoliae flos can be used as anti-asthmatic agents (Korean Patent No. 0263439). Moreover, macelignan is a typical lignan compound found in Myristica fragrans (Tuchinda P. et al., Phytochemistry, 59: 169-173, 2002), and was reported to have various activities, such as the activation of caspase-3 inducing apoptosis (Park B. Y. et al., Biol. Pharm. Bull., 27(8): 1305-1307,2004), and antibacterial activity. However, there is still no report on the use of lignan compounds, including macelignan as brain disease-treating agents.

### DETAILED DESCRIPTION OF THE INVENTION

Technical Problem

Accordingly, the present inventors have conducted a long-term investigation to find a naturally derived compound capable of treating brain disease-treating activity and, as a result, found that a lignan compound isolated and purified from a Myristica fragrans extract shows excellent effects for treating and preventing a brain disease, thereby completing the present invention.

It is an object of the present invention to provide the novel use of the Myristica fragrans extract or lignan compounds isolated and purified therefrom for treating or preventing a brain disease.

Technical Solution

To achieve the above-mentioned object, in one aspect, the present invention provides a pharmaceutical composition for treating or preventing a brain disease, comprising a lignan compound represented by Formula I or a pharmaceutically acceptable salt thereof as an active ingredient:

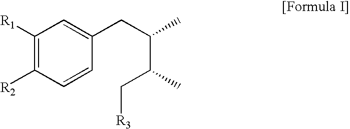

wherein R₁ and R₂ are each independently a C₁₋₅ alkoxy group or a hydroxyl group, and R₃ is

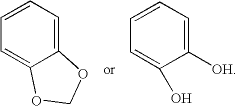

In another aspect, the present invention provides a method for treating or preventing a brain disease, comprising administering to a subject in need thereof an effective amount of a lignan compound represented by Formula I.

In still another aspect, the present invention provides a method for inhibiting a brain cell death, comprising administering to a subject in need thereof an effective amount of a lignan compound represented by Formula I.

In yet another aspect, the present invention provides the use of a lignan compound represented by Formula I for production of a brain disease-treating agent.

In a further aspect, the present invention provides the use of a lignan compound represented by Formula I for production of a brain cell death inhibitor.

In another aspect, the present invention provides a pharmaceutical composition for treating or preventing a brain disease, comprising a water or C₁-C₆ organic solvent extract of Myristica fragrans as an active ingredient.

In additional aspect, the present invention provides a method for treating or preventing a brain disease, comprising administering to a subject in need thereof an effective amount of a water or C₁-C₆ organic solvent extract of Myristica fragrans.

In still another aspect, the present invention provides a method for inhibiting a brain cell death, comprising administering to a subject in need thereof an effective amount of a water or C₁-C₆ organic solvent extract of Myristica fragrans.

In yet another aspect, the present invention provides the use of a water or C₁-C₆ organic solvent extract of Myristica fragrans for production of a brain disease-treating agent.

In a further aspect, the present invention provides the use of a water or C₁-C₆ organic solvent extract of Myristica fragrans for production of a brain cell death inhibitor.

As used herein, the term “effective amount” refers to the amount of the inventive lignan compound or Myristica fragrans extract, which can effectively inhibit the brain cell death and treat and/or prevent a brain disease when being administered to a subject.

Also, as used herein, the term “subject” encompasses mammals, particularly animals including human beings. The subject may be a patient in need of treatment.

Hereinafter, the present invention will be described in detail.

The present invention is characterized by providing the novel use of a Myristica fragrans extract and a lignan compound isolated and purified therefrom.

The lignan compound according to the present invention is represented by Formula I:

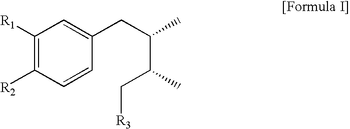

wherein R₁ and R₂ are independently a C₁₋₅ alkoxy group or a hydroxyl group, and R3 is

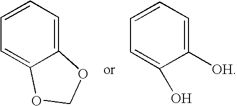

In the present invention, the preferable lignan compound may be macelignan of Chemical Formula I, i.e., [(8R,8′S)-7-(3,4-methylenedioxyphenyl)-7′-(4-hydroxy-3-methoxyphenyl)-8,8′-dimethylbutane)], wherein R₁ is a methoxy group, R₂ is a hydroxyl group, and R₃ is

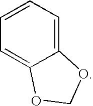

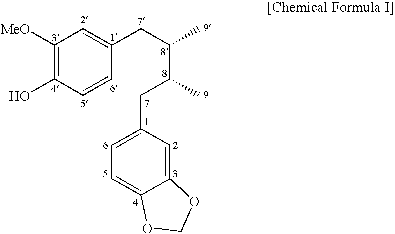

The lignan compound according to the present invention may be used in the form of a salt, and preferably a pharmaceutically acceptable salt. Preferably, the salt is the acid-addition salt formed by a pharmaceutically acceptable free acid. The free acid used in the present invention may be organic acids and inorganic acids. The organic acids include, but are not limited to, citric acid, acetic acid, lactic acid, tartar acid, maleic acid, fumaric acid, formic acid, propionic acid, oxalic acid, trifluoroacetic acid, benzoic acid, gluconic acid, m-sulfonic acid, glycolic acid, succinic acid, 4-toluene sulfonic acid, glutamic acid and aspartic acid. Also, the inorganic acids include, but are not limited to, hydrochloric acid, bromic acid, sulfuric acid and phosphoric acid.

The lignan compound of the present invention can be obtained from a plant or part of a plant according to any conventional method for extracting and isolating substance. Stems, roots or leaves are suitably dehydrated and macerated or only dehydrated in order to obtain the desired extract, which is then purified using any conventional purification method known to a person skilled in the art. Moreover, synthetic compounds or their derivatives corresponding to the lignan compound represented by Formula I are generally commercially available substances or they may be chemically manufactured using any known synthetic method.

The lignan compound of the present invention represented by Formula I may be isolated and purified from Myristica fragnance Houtt (Jung Yun Lee et al., Kor. J. Pharmacogn. 21(4):270-273, 1990). Preferably, it may be isolated and purified from nutmeg or aril. The nutmeg refers to the ripe fruit of Myristica fragnance or a seed contained in the fruit. Moreover, the lignan compound of the present invention may also be isolated and purified from oil obtained by squeezing nutmeg. Also, it may be isolated and purified from Myristica argentea Warb, another member of the Myristicaceae family (Filleur, F. et al., Natural Product Letters, 16: 1-7, 2002). In addition, it may also be isolated and purified from Machilus thunbergii (Park B. Y. et al., Biol. Pharm. Bull., 27(8): 1305-1307,2004), and Leucas aspera (Sadhu, S. K. et al., Chem. Pharm. Bull., 51(9): 595-598, 2003).

An extraction solvent for isolating the lignan compound of the present invention may be water or a C₁-C₆ organic solvent. Preferred examples of the extraction solvent may include purified water, methanol, ethanol, propanol, isopropanol, butanol, acetone, ether, benzene, chloroform, ethyl acetate, methylene chloride, hexane, cyclohexane, petroleum ether and the like, which can be used alone or a mixture thereof. More preferably, methanol or hexane may be used. The isolation and purification of the lignan compound of the present invention from the extract of Myristica fragnance may be performed by, for example, column chromatography and high-performance liquid chromatography (HPLC), packed with various synthetic resins, such as silica gel or activated alumina, or a combination of. However, the method for extracting, and isolating and purifying the active ingredient needs not to be limited to these chromatography techniques.

As such, the lignan compound of the present invention may be used in the form of a purely isolated and purified compound or in the form of an extract containing the compound. For example, as described above, the lignan compound of the present invention may be used in the form of an extract of the seed, fruit or aril of Myristica fragnance, or in the form of oil obtained by squeezing the seed of Myristica fragnance. As described above, the extract can be obtained by extracting Myristica fragnance with water or a C₁-C₆ organic solvent. Preferably, the extract may be an extract of the seed of Myristica fragnance, namely, a nutmeg extract.

The reactive oxygen species, a substance causing the oxidative toxicity in vivo, induces lipid peroxidation that is a component of a cell membrane, thereby destroying the bio-protection and signal transfer system of the cell membrane, and also induces oxidative damage of DNA, destruction of a red blood cell and protein peroxidation, thereby lowering the function of various enzymes in vivo. Through them, the reactive oxygen species are known to cause various diseases such as cancer, brain diseases including cerebral apoplexy and Parkinson's disease, heart disease, ischemia, arteriosclerosis, skin disease, gastric disease, inflammation, rheumatism and autoimmune disease, as well as aging. It is suggested that the reactive oxygen species is a main factor of Alzheimer's disease (Maccioni et al., Arch. Med. Res., 32:367-281, 2001). Therefore, in an example of the present invention, the inhibition of the reactive oxygen species production by the lignan compound of the present invention was investigated. As a result, it was shown that the lignan compound of the present invention inhibited the production of the reactive oxygen species caused by glutamate in a cell line HT22 derived from the hippocampus in the brain as well as by BSO (buthionine sulfoxide) in the cultured neuron of the hippocampus in a concentration dependent manner (See FIGS. 7 and 8).

The lipid peroxidation is an index showing the brain damage by oxidative stresses (Sewerynek et al., Neuroscience Letter, 195:203-205, 1995). The hydrogen peroxide is dissociated into water and oxygen. In this process, a hydroxyl free radical is produced. The free radical causes DNA damage, protein carbonylation and lipid peroxidation. Therefore, in another example of the present invention, the inhibition of the hydrogen peroxide-mediated lipid peroxidation by the lignan compound of the present invention was investigated. As a result, it was shown that the lignan compound of the present invention inhibited the lipid peroxidation in a concentration dependent manner (See FIG. 9).

In another example of the present invention, it was investigated whether the lignan compound of the present invention showed cytotoxicity itself. As a result, it was shown that the lignan compound of the present invention did not show cytotoxicity even at a concentration of 10 μM (See FIG. 10).

Another reason for the brain cell death is apoptosis via glutamate as an excitatory neurotransmitter and its receptor (Olney, J. W., Int Rev. Neurobiol., 27:337-362, 1985). Therefore, in another example of the present invention, it was investigated whether the lignan compound of the present invention inhibited the apoptosis of brain cell induced by the glutamate. As a result, it was shown that the apoptosis of brain cell induced by the glutamate was inhibited by the lignan compound of the present invention in a concentration dependent manner (See FIG. 11).

Additionally, epidemiological evidence that nonsteroidal anti-inflammatory drugs such as ibuprofen delayed the progress of Alzheimer's disease was reported (McGeer and McGeer, Exp. Gerontol., 33:371-378, 1998). In particularly, it was shown that administration of ibuprofen into a mouse having a mimic Alzheimer's disease delayed the progress of the disease (Lim et al., J. Neurosci., 20″5709-5714, 2000). Therefore, a compound having the anti-inflammatory activity as well as antioxidative activity is highly effective on the treatment and prevention of brain disease. Accordingly, in another example of the present invention, anti-inflammatory activity of the lignan compound of the present invention was investigated by treating a microglia as a brain immune cell with LPS (lipopolysaccharide). As a result, it was shown that the lignan compound of the present invention significantly reduced the expression and production of various immune mediators such as IL-6, TNF-α, NO, iNOS and COX-2 induced by LPS (See FIGS. 12 to 15).

As mentioned above, the lignan compound of the present invention has an excellent antioxidative effect inhibiting lipid peroxidation and production of reactive oxygen species, an excellent brain cell protection effect inhibiting the apoptosis of a brain cell, and an excellent anti-inflammatory effect. Therefore, the present invention provides a pharmaceutical composition for treatment and prevention of a brain disease comprising the lignan compound represented by formula I or a pharmaceutically acceptable salt thereof as active ingredients. Additionally, the present invention provides a pharmaceutical composition for treatment and prevention of a brain disease comprising the Myristica fragnance extract. The preparation of the Myristica fragnance extract is described above.

Additionally, the present invention provides a method and use for treatment and prevention of a brain disease comprising administering to a subject in need thereof an effective amount of the lignan compound represented by formula I or the Myristica fragnance extract.

Additionally, the present invention provides a method and use for inhibition of a brain cell death comprising administering to a subject in need thereof an effective amount of the lignan compound represented by formula I or the Myristica fragnance extract.

The composition of the present invention can be administered in an oral or parenteral manner and used in the form of common drug formulations when clinical administration. The common drug formulations may be prepared using diluents or excipients such as fillers, thickeners, binders, wetting agents, disintegrants and surfactants. Solid formulations for oral administration include tablets, pills, powders, granules and capsules, and are prepared by combining the lignan compound or the Myristica fragnance extract with at least one excipient, for example, starch, calcium carbonate, sucrose, lactose or gelatin. Also, except the simple excipient, lubricant such as magnesium stearate or talc may be used. Examples of liquid formulations for oral administration include suspensions, liquid preparations, emulsions and syrups. The liquid formulations may comprise a simple diluent such as water, liquid paraffin, and various excipients, for example, humectants, sweeteners, aromatic agents and preservatives. Examples of pharmaceutical formulations for parenteral administration include sterilized aqueous solutions, non-aqueous solutions, suspensions, emulsions, freeze-dried preparations, ointments and creams. The non-aqueous solutions and suspensions may be prepared using propylene glycol, polyethylene glycol, vegetable oils such as olive oil, and injectable esters such as ethyloleate.

Also, the composition of the present invention may be administered by parenteral routes, including subcutaneous, intravenous, intramuscular or intraperitoneal injection. For parenteral administration, the lignan compound represented by Formula I or the Myristica fragnance extract may be mixed with a stabilizer or buffer in water to prepare a solution or suspension, which may then be formulated as a unit dosage form of ampules or vials. The dosage units can contain, for example, 1, 2, 3, or 4 times of an individual dose or ½, ⅓ or ¼ times of an individual dose. Preferably, an individual dose contains the amount of an effective drug which is administered in one dosage and which usually corresponds to a whole, a half, a third or a quarter of a daily dose.

The lignan compound of the present invention represented by Formula I or the Myristica fragnance extract can be administered in an effective dosage of 0.1-50 mg/kg, and preferably 1-10 mg/kg, 1-3 times a day. The dosage of the lignan compound represented by Formula I or the Myristica fragnance extract may vary depending on, for example, the body weight, age, sex, health condition, diet, time of administration, method of administration, excretion rate and disease severity for a certain patient.

The lignan compound of the present invention was tested for toxicity in oral administration to rats, and as a result, it was observed that the 50% lethal dose (LD50) was more than 2,000 mg/kg (Data is not shown).

The term “brain disease” to which the pharmaceutical composition of the present invention is applicable refers to a disease resulted from the death or degeneration of brain cells caused by oxidative stresses by lipid oxidation, reactive oxygen species and/or free radicals; excitatory toxicity by glutamate; and/or apoptosis. The examples of the brain disease include, for example, degenerative brain disease such as dementia, mild cognitive impairment, Parkinson's disease and Huntington's disease, ischemic brain disease such as cerebral apoplexy and convulsive brain disease such as epilepsy. In particular, the brain diseases may include, but are not limited to, dementia, Parkinson's disease, cerebral apoplexy, Huntington's disease, Creutzfeldt-Jakob disease, Pick's disease, amyotrophic lateral sclerosis(ALS), Parkinson-ALS-dementia complex, Wilson's disease, progressive supranuclear palsy, mild cognitive impairment and epilepsy, wherein the dementia include all of senile dementia,

Alzheimer type dementia, vascular dementia, alcoholic dementia and thalamic dementia.

### BRIEF DESCRIPTION OF THE DRAWINGS

FIG. 1 shows a process of isolating a lignan compound from Myristica fragrans.

FIG. 2 shows the ¹³C-NMR spectrum of the lignan compound of the present invention.

FIG. 3 shows the ¹H-NMR spectrum of the lignan compound of the present invention.

FIG. 4 shows the ¹H—¹H COSY spectrum of the lignan compound of the present invention.

FIG. 5 shows the ¹H—¹³C HMBC spectrum of the lignan compound of the present invention.

FIG. 6 shows the EI-Mass spectrum of the lignan compound of the present invention.

FIG. 7 is the graph showing the inhibitory effect of the lignan compound of the present invention on the reactive oxygen species production by glutamate in a cell HT-22.

FIG. 8 is the graph showing the inhibitory effect of the lignan compound of the present invention on the reactive oxygen species production by BSO in a cultured hippocampus cell.

FIG. 9 is the graph showing the inhibitory effect of the lignan compound of the present invention on the lipid peroxidation of the brain tissue caused by hydrogen peroxide for various concentrations.

FIG. 10 is the graph showing the cytotoxicity effect of the lignan compound of the present invention.

FIG. 11 is the graph showing the inhibitory effect of the lignan compound of the present invention on the apoptosis induced by glutamate for various concentrations.

FIG. 12 is the graph showing the inhibitory effect of the lignan compound of the present invention on IL-6 in a cultured microglia of the brain.

FIG. 13 is the graph showing the inhibitory effect of the lignan compound of the present invention on TNF-α in a cultured microglia of the brain.

FIG. 14 is the graph showing the inhibitory effect of the lignan compound of the present invention on NO in a cultured microglia of the brain.

FIG. 15 shows the results for the inhibitory effect of the lignan compound of the present invention on the expression of proteins iNOS and COX-2 induced by LPS in a cultured microglia of the brain.

A: an analysis result of Western blotting; and

B: a graph quantitatively showing the inhibitory effect of the lignan compound of the present invention on the expression of the proteins iNOS and COX-2.

FIG. 16 shows the results of LC/MS analysis using macelignan of the present invention.

### BEST MODE FOR CARRYING OUT THE INVENTION

Hereinafter, the present invention will be described in detail by examples. It is to be understood, however, that these examples are for illustrative purpose only and are not intended to limit the scope of the present invention.

### EXAMPLE 1

Isolation and Purification of Lignan Compound from Myristica fragrans

<1-1> Isolation and Purification of Lignan Compound

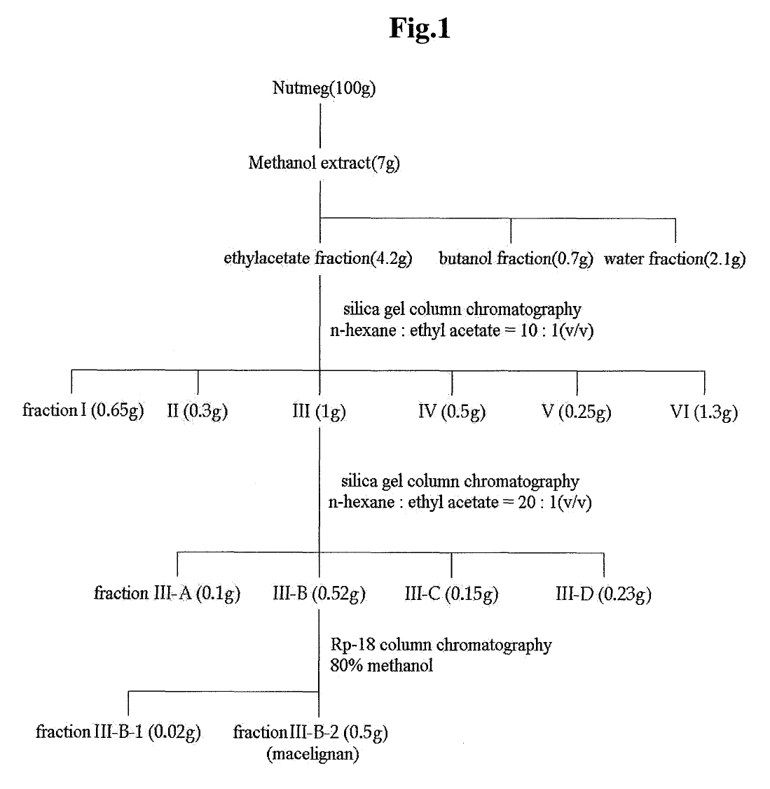

To 100 g (dry weight) of dried and crushed nutmeg, 400 ml of 75 vol. % methanol was added, and the solution was left to stand at room temperature for 2 days. The solution was then filtered through Whatman filter paper No. 2. The filtration step was repeated two times. The methanol filtrate was concentrated under vacuum and lyophilized to prepare 7 g of a methanol crude extract of nutmeg. The methanol crude extract was fractionated sequentially with ethyl acetate, butanol and water to obtain 4.2 g of an ethyl acetate fraction. The ethyl acetate fraction was eluted by silica gel column chromatography (Merck Kieselgel 66; 70-230 mesh) with a mixed solvent of hexane and ethyl acetate (10:1 v/v) to obtain 1.0 g of fraction III. The solvent was completely removed with a vacuum rotary evaporator to prepare a crude extract of nutmeg. The fraction III was then eluted by silica gel column chromatography (Merck Kieselgel 66; 70-230 mesh) with a mixed solvent of hexane and ethyl acetate (20:1 v/v) to obtain 0.52 g of fraction III-B. The fraction III-B was then eluted by Rp-18 column chromatography (Merck LiChroprep; 25-40 μm) with 80% methanol to obtain 0.5 g of single compound fraction III-B-2. This isolation process was shown in FIG. 1.

<1-2> Analysis of Structure

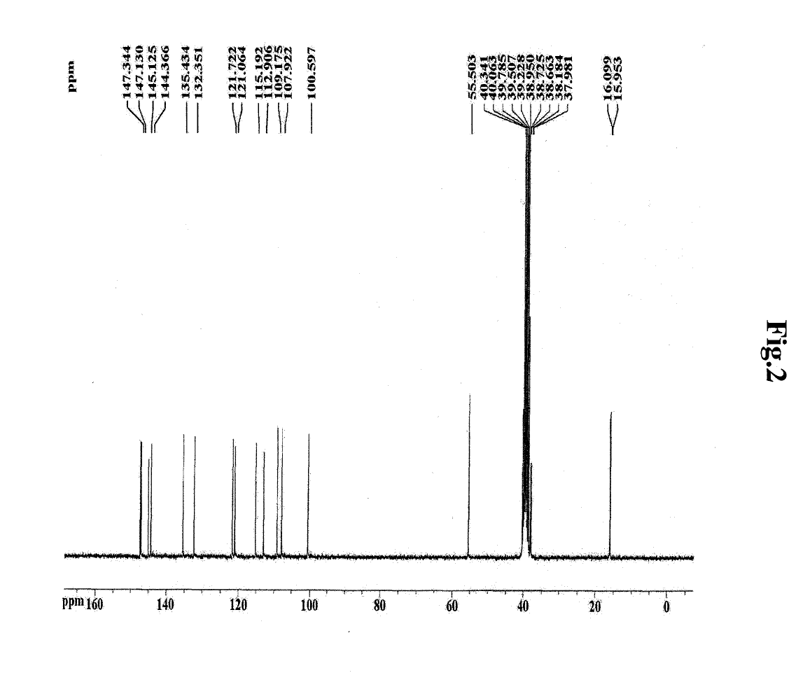

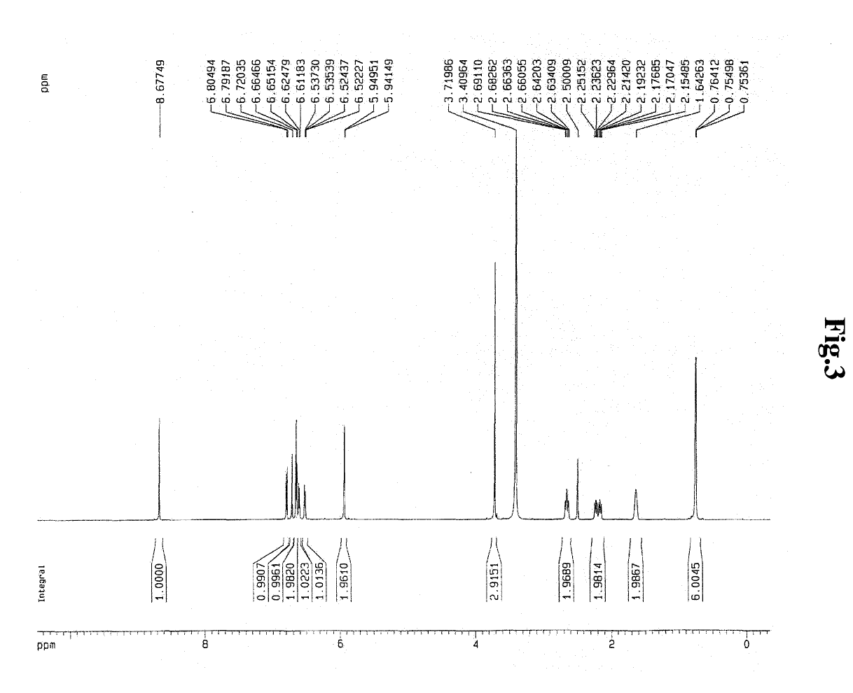

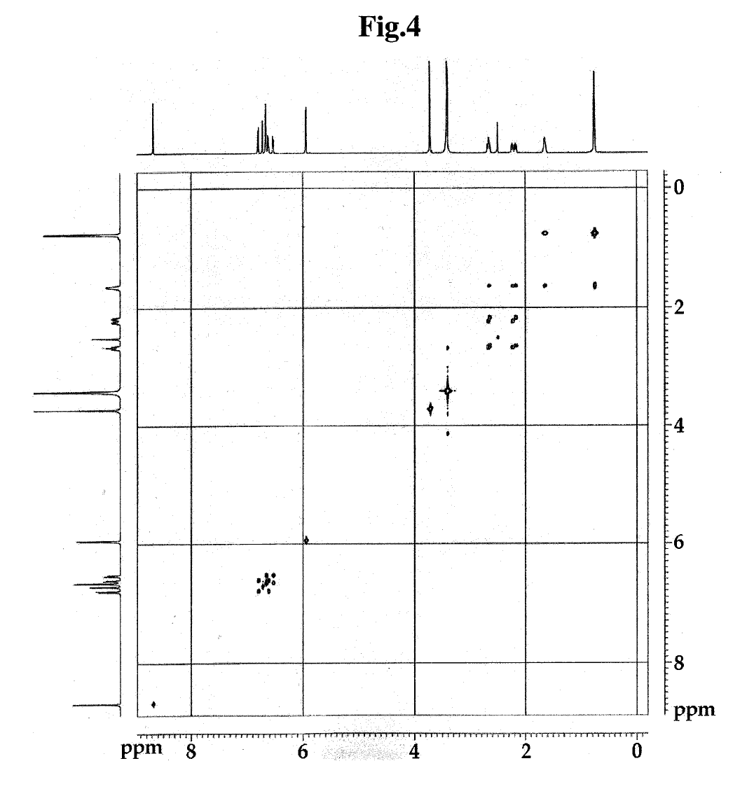

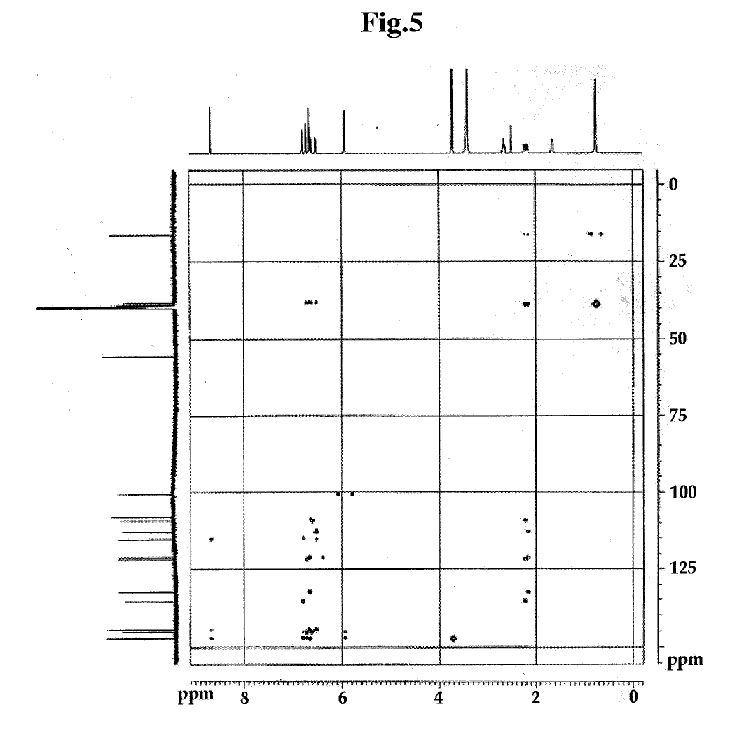

To determine the structure of the isolated single compound fraction III-B-2, the ¹H-NMR spectrum and ¹³C-NMR spectrum were analyzed at 600 MHz and 150 MHz, respectively, in DMSO solvent. The results were shown in FIGS. 2 and 3, respectively. To determine ¹H—¹H correlation and ¹H—¹³C correlation on the basis of the results of the ¹³C-NMR and ¹H-NMR spectrum analyses, the ¹H—¹H COSY spectrum and ¹H—¹³C HMBC spectrum were analyzed. The results were shown in FIGS. 4 and 5, respectively. The results of the ¹H-NMR, ¹³C-NMR, ¹H—¹H COSY and ¹H—¹³C HMBC spectrum analyses were collectively analyzed and the results were shown in Table 1 below.

TABLE 1

| Position | ¹³C-NMR | ¹H-NMR | ¹H-¹H COSY | ¹H-¹³C HMBC |
|---|---|---|---|---|
| 1 | 135.4 | | | |
| 2 | 109.2 | 6.72 brs | | C-7, C-6, C-4, C-3 |
| 3 | 147.3 | | | |
| 4 | 145.1 | | | |
| 5 | 107.9 | 6.79 d(7.8) | 6.61 | C-6, C-4, C-3, C-1 |
| 6 | 121.7 | 6.61 dd(7.8) | 6.79 | C-7, C-5, C-4, C-2, C-1 |
| 7 | 38.2 | 2.23 dd(13.2, 9.3) | 1.64, 2.66 | C-8, C-6, C-2, C-1 |
| | | 2.66 dd(13.2, 4.8) | 1.64, 2.23 | C-9, C-8, C-6, C-2, C-1 |
| 8 | 38.7 | 1.64 brs | 0.75, 2.23, 2.66 | C-7 |
| 9 | 16.0 | 0.75 d(6.3) | 1.64 | C-8, C-7 |
| 1′ | 132.4 | | | |
| 2′ | 112.9 | 6.66 brs | | C-7′, C-6′, C-4′, C-3′ |
| 3′ | 147.1 | | | |
| 4′ | 144.4 | | | |
| 5′ | 115.2 | 6.66 d(7.9) | 6.53 | C-6′, C-4′, C-3′, C-1′ |
| 6′ | 121.0 | 6.53 d(7.9, 1.1) | 6.66 | C-7′, C-5′, C-4′, C-2′, C-1′ |
| 7′ | 38.0 | 2.17 dd(13.2, 9.3) | 1.64, 2.66 | C-8′, C-6′, C-2′, C-1′ |
| | | 2.66 dd(13.2, 4.8) | 1.64, 2.17 | C-9′, C-8′, C-6′, C-2′, C-1′ |
| 8′ | 38.7 | 1.64 brs | 0.75, 2.17, 2.66 | C-7′ |
| 9′ | 16.1 | 0.75 d(6.3) | 1.64 | C-8′, C-7′ |
| OMe | 55.5 | 3.72(s) | | |
| O—CH₂—O | 100.6 | 5.95 d(4.8) | | C-3, C-4 |

<1-3> Mass Analysis

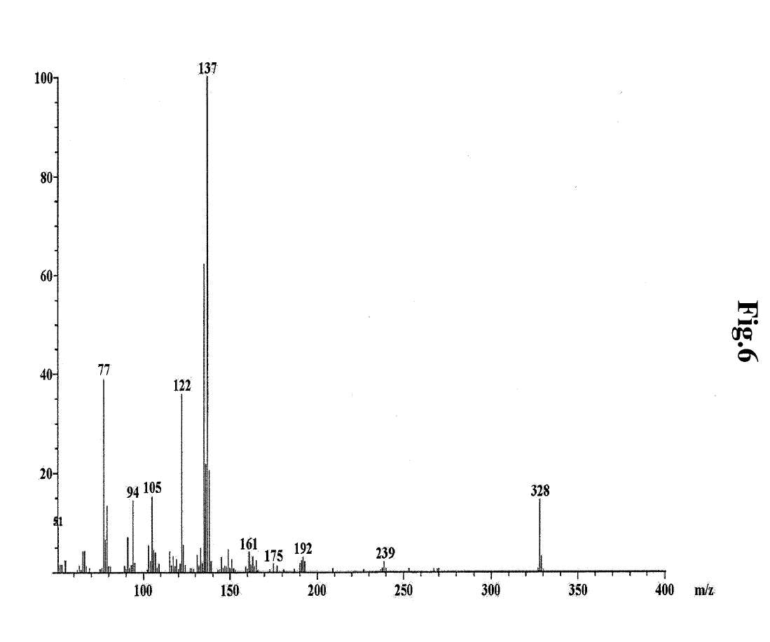

The results of El/MS analysis for the mass analysis of the above-isolated single compound III-B-2 were shown in FIG. 6. In the EI/MS analysis, [M]⁺ was observed at m/z 328, indicating that the isolated compound has a molecular weight of 328 dalton and a molecular formula of C₂₀H₂₄O₄.

<1-4> Optical Rotation Measurement

The optical rotation was measured by dissolving 20 mg of the above-isolated single compound III-B-2 in 2 ml of chloroform(CHCl₃), and analyzing with an automatic polarimeter(APIII-589, Rodulph, N.J., USA). As a result, the optical rotation ([α]_{D}) was +4.0 (CHCl₃, c=1.0).

The results of the ¹H-NMR, ¹³C-NMR, ¹H—¹H COSY, ¹H—¹³C HMBC, EI/MS and [α]_{D} analyses were analyzed comparatively with the previously reported study results (Woo, W. S. et al., Phytochemistry, 26: 1542-1543, 1987). As a result, it was found that the isolated single compound was macelignan represented by Chemical Formula I:

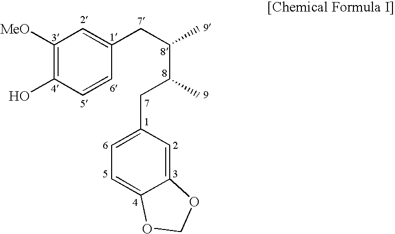

### EXAMPLE 2

Examination of Reactive Oxygen Species-Inhibitory Effect of Lignan Compound of the Present Invention

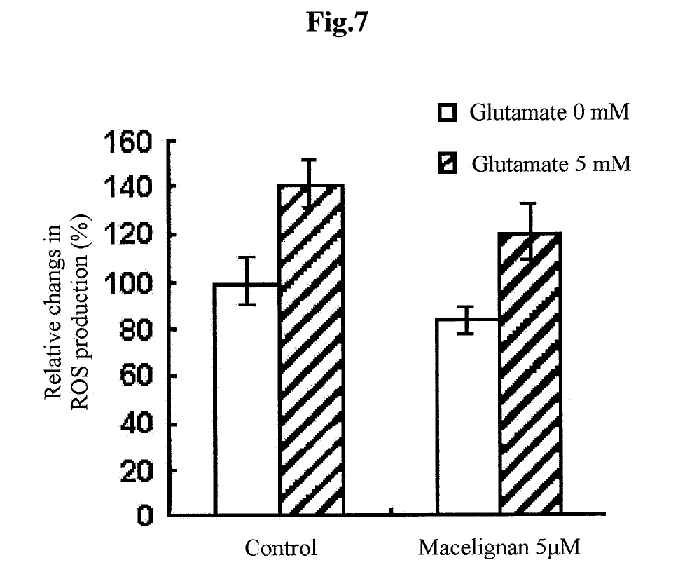

A cell line HT-22 (obtained from Dr. David Schubert) derived from the hippocampus playing a part in memory of the brain was treated with 5 mM of glutamate and 5 μM of the lignan compound of the present invention simultaneously for 8 hours. As a control, the cell line HT-22 was not treated with the lignan compound of the present invention. The treated cell line HT-22 was then treated with CM-H₂DCFDA (chloromethyl derivative of dichlorodihydrofluorescein diacetate, Molecular Probes) to analyze the production of reactive oxygen species. As a result, shown in FIG. 7, the lignan compound of the present invention inhibited the production of reactive oxygen species induced by glutamate. Additionally, in the control which was not treated with glutamate, the lignan compound of the present invention significantly inhibited the production of reactive oxygen species occurred naturally in a cell.

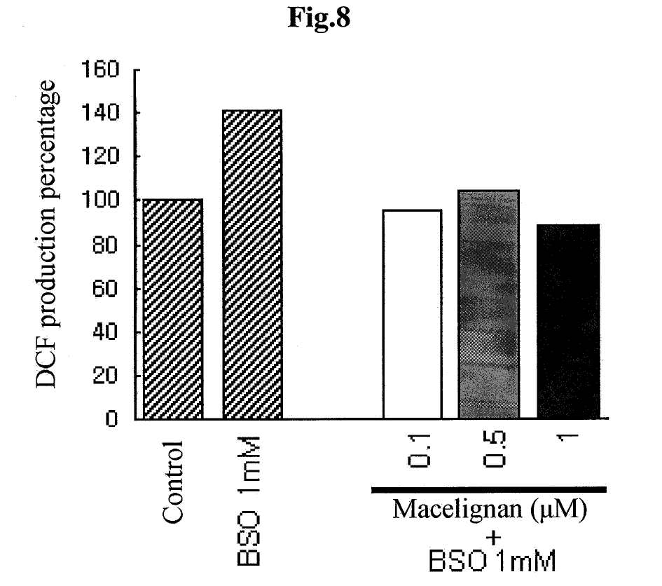

Then, the antioxidant effect of the lignan compound of the present invention in the tissue-cultured hippocampus neuron was analyzed. For this analysis, the hippocampus of a fetus extracted from a pregnant (18 days) mouse was cultured in neurobasal medium (Gibco BRL) containing B-27 supplements and 2 mM L-glutamine for 10 days. To induce oxidative stress to the tissue-cultured hippocampus neuron, it was treated with 1 mM BSO for 8 hours. Here, the tissue-cultured hippocampus neuron was also treated with each concentration (0.1, 0.5 and 1 μM) of the lignan compound of the present invention simultaneously to analyze the reactive oxygen species-inhibitory effect of the lignan compound of the present invention. The control was not treated with BSO. Then, the amount of reactive oxygen species produced was measured by a method known in the art (Jung, Y. S., Biochem Biophys Res Commun., 320(3):789-94, 2004) using 10 μM DCFDA(molecular probes). As a result, shown in FIG. 8, the lignan compound of the present invention inhibited the production of reactive oxygen species in the tissue-cultured hippocampus neuron in the similar level as the control.

### EXAMPLE 3

Examination of Lipid Peroxidation-Inhibitory Effect of the Lignan Compound of the Present Invention

After anesthesia of a rat, the tissue of the rat was perfused with 0.9% physiological saline solution containing EDTA. The brain was then extracted from the rat and washed with ice-cold 20 mM Tris-HCl (pH 7.4). After removing water, the weight of the brain was measured. The brain was mixed with 0.1 g/ml of ice-cold 20 mM Tris-HCl (pH 7.4) and homogenized. The homogenized brain was centrifuged and the supernatant was collected. 40 μl of the supernatant was mixed with 40 mM of hydrogen peroxide to induce lipid peroxidation. Simultaneously, different concentration (0.5, 1, 5 and 10 μM) of the lignan compounds isolated from the Example 1 were added into the supernatant, respectively. After incubation of the supernatant in water (37° C.) for 30-60 minutes, 162.5 μl of R1 solution (lipid peroxidation assay kit, Cat. No. 437634, Calbiochem) and 37.5 μl of R2 solution (lipid peroxidation assay kit, Cat. No. 437634, Calbiochem) were added into the cultured supernatant, and the supernatant was further incubated at 45° C. for 40 minutes. Then, the absorbance of the incubated medium was measured at 586 nm to quantify the lipid peroxidation.

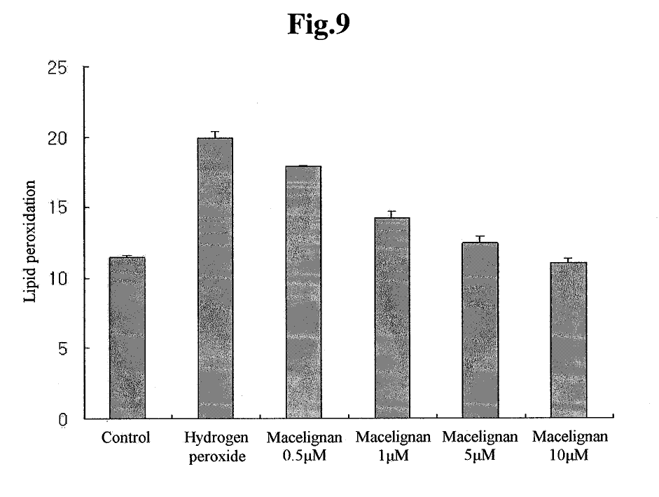

As a result, shown in FIG. 9, it was observed that the lipid peroxidation induced by hydrogen peroxide was dose-dependently inhibited by the lignan compound of the present invention. Especially, 10 μM of the lignan compound of the present invention inhibited the lipid peroxidation in the similar level as the control in which the lipid peroxidation was not induced by hydrogen peroxide.

### EXAMPLE 4

Examination of Cytotoxicity Effect of Lignan Compound of the Present Invention

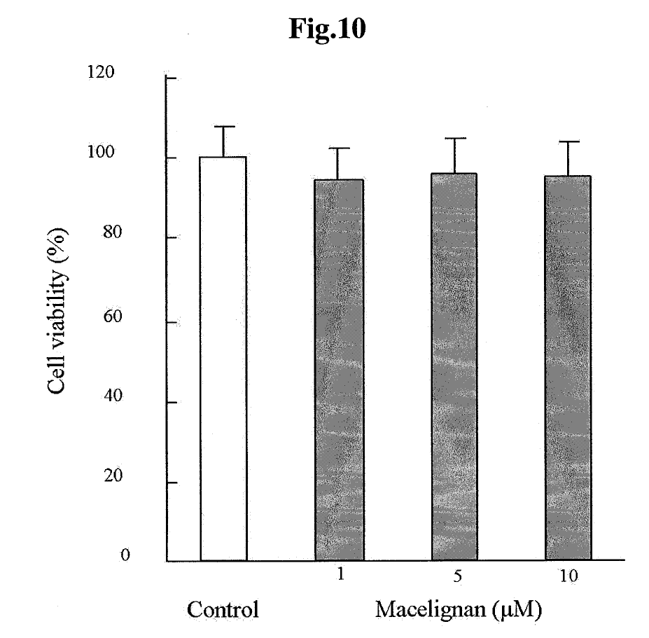

In order to examine the cytotoxicity effect of macelignan itself, a cell line HT-22 derived from the hippocampus was treated with macelignan at concentrations (1, 5 and 10 μM) for 24 hours. As a result, shown in FIG. 10, the macelignan of the present invention did not induce cytotoxicity at even 10 μM.

### EXAMPLE 5

Examination of Brain Cell Apoptosis-Inhibitory Effect of Lignan Compound of the Present Invention

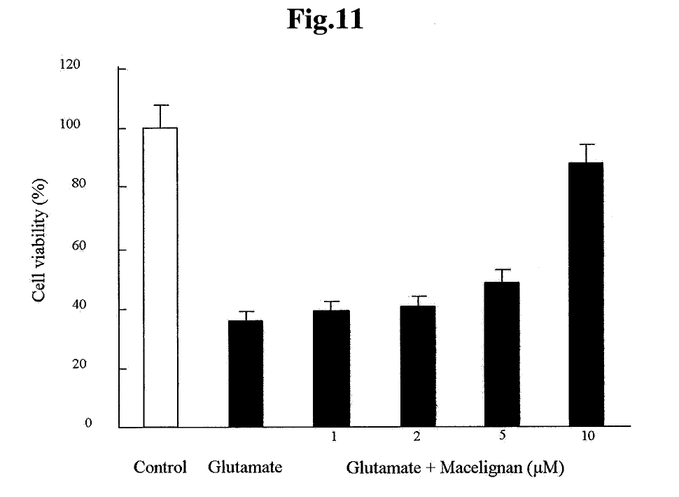

A cell line HT-22 derived from the hippocampus was treated with 5 mM glutamate for 24 hours to induce apoptosis. As a control, the cell line HT-22 was not treated with glutamate. Then, the cell line HT-22 was treated with the lignan compound of the present invention at concentrations (1, 2, 5 and 10 μM) for 24 hours. The cell death was analyzed with WST-1(Roche). As a result, shown in FIG. 11, it was observed that the lignan compound of the present invention dose-dependently inhibited the brain cell apoptosis induced by glutamate.

### EXAMPLE 6

Examination of Anti-Inflammatory Effect of Lignan Compound of the Present Invention

<6-1> Pro-Inflammatory Cytokine-Inhibitory Effect

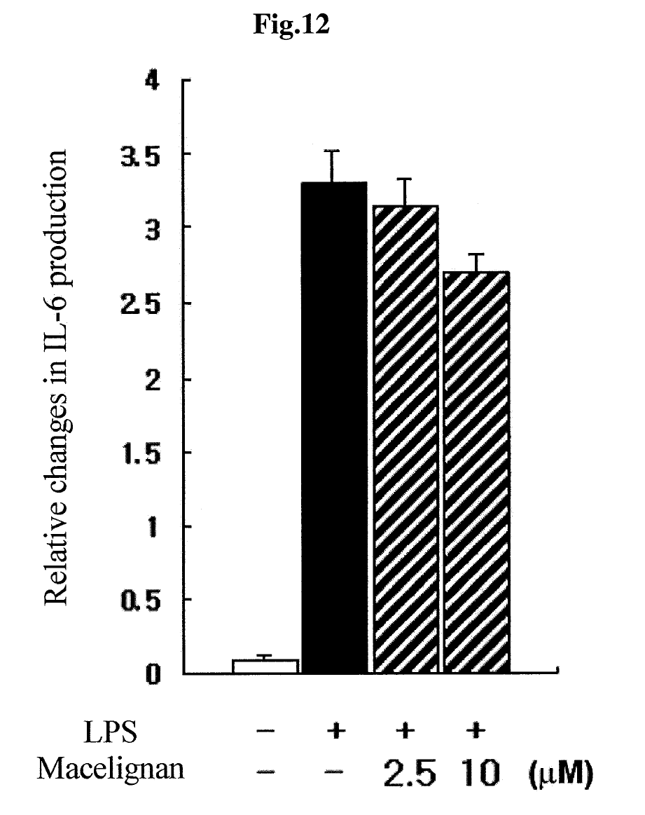

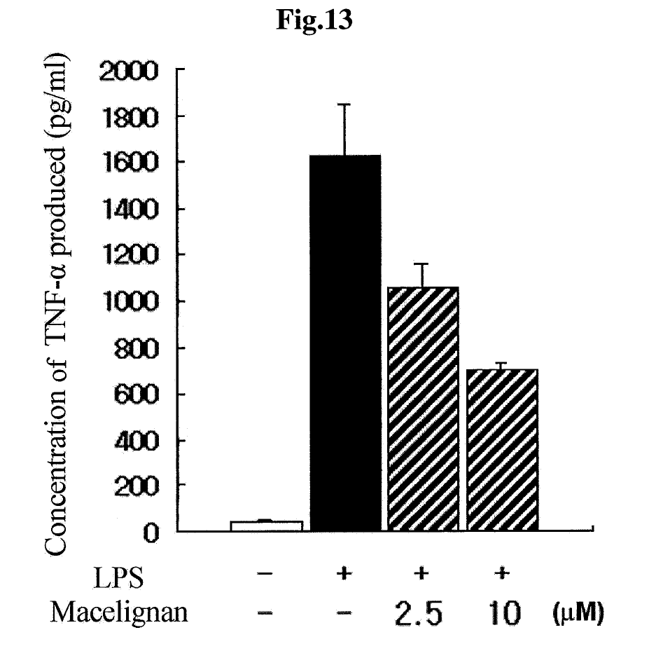

Microglia is the only cell originated from the mesoderm in the central nervous system, and is significantly increased when an inflammation reaction is occurred in the brain tissue (Streit, W. J. Prog. Neurobiol., 57:563-581, 1999). When the microglia is activated by LPS, it synthesizes and secretes various pro-inflammatory cytokines such as IL-1, IL-6 and TNF-α (Chen, S. Neurobiol. Aging, 17:781-787, 1996). Therefore, in order to examine the anti-inflammatory effect of the lignan compound of the present invention, the effect of the lignan compound of the present invention on the production of IL-6 and TNF-α in the activated microglia. First, only the neocortex was isolated from the brain of 1 day-old rat. Then, a microglia-astroglia mixture was prepared according to a method known in the art (Kim, H. Y. et al., J. Immunol., 171:6072-6079, 2003). The microglia-astroglia mixture was then divided at a ratio of 4 flasks/1 head and cultured in MEM medium containing 10% FBS in a 75 cm² flask for 2 weeks. The microglia was separated from the cultured cells and was cultured in MEM medium containing 5% FBS for 24 hours. The cultured microglia was then washed with the serum-free medium 2 times, and treated with 1 μg/ml of LPS and 2.5 and 10 μM of the lignan compound of the present invention for 24 hours. Then, the amount of IL-6 and TNF-α secreted into the cell cultured medium was measured with the solid-phase ELISA system (RPN2742 for IL-6, RPN2744 for TNF-α, Amersham Bioscience). For this analysis, 50 μl of the supernatant of the cultured medium of microglia and 50 μl of the standard of each material (purely isolated and quantified IL-6 or TNF-α) were added into a 96-well plate coated with an antibody specific to mouse IL-6 and TNF-α. After 2 hours of the reaction at room temperature, each well was washed with a washing buffer (Amersham Bioscience) 3 times. 100 μM of the antibody specific to IL-6 or TNF-α treated with biotin was added into each well and incubated at room temperature for 1 hour. Each well was washed with a washing buffer 3 times. 100 μl of streptavidin solution (Amersham Bioscience) coupled with HRP was added into each well, and incubated at room temperature for 30 minutes. Then, 100 μl of a stop solution (Amersham Bioscience) was added into each well to stop the reaction, and the absorbance of the solution in each well was measured with a microreader at 450 nm. As a result, shown in FIGS. 12 and 13, the production of pro-inflammatory cytokines (IL-6 and TNF-α) induced by LPS was dose-dependently inhibited by the lignan compound of the present invention. Especially, the inhibitory effect on the production of TNF-α was higher.

<6-2> NO Production-Inhibitory Effect in Microglia Activated by LPS

When the microglia is activated, the expression of NO as a mediator of nerve transmission and immune response is induced (Liu, B. et al., Ann. N.Y. Acad. Sci., 962:318-331, 2002). Therefore, the effect of the lignan compound of the present invention on the NO production in microglia was examined. First, only the neocortex was isolated from the brain of 1 day-old mouse. Then, a microglia-astroglia mixture was prepared according to a method known in the art (Kim, H. Y. et al., J. Immunol., 171:6072-6079, 2003). The microglia-astroglia mixture was then divided at a ratio of 4 flasks/1 head and cultured in MEM medium containing 10% FBS in a 75 cm² flask for 2 weeks. The microglia was separated from the cultured cells and cultured in MEM medium containing 5% FBS for 24 hours. The tissue-cultured microglia was inoculated into MEM medium containing 5% FBS at a concentration of 1.5×10⁴ cells/well and cultured in a 96-well plate. After 1 day of cultivation, the microglia was treated with 1 μg/ml of LPS (Sigma) to induce activation of the microglia. Simultaneously, the microglia was also treated with 2.5 and 10 μM of the lignan compound of the present invention together with LPS, and reacted for 16 or 24 hours. Then, the cell-cultured medium was collected and NO production was measured. The NO production was analyzed by measuring the amount of nitrite as a stable metabolite of NO in the cell-cultured medium using Griess reagent kit (Molecular Probe). The measuring method is as follows: 150 μl of the cell-cultured medium was mixed with 20 μl of Griess reagent and 130 μl of water, and incubated in a microplate at room temperature for 30 minutes. Then, the absorbance of the resulting solution was measured with a microplate reader at 548 nm.

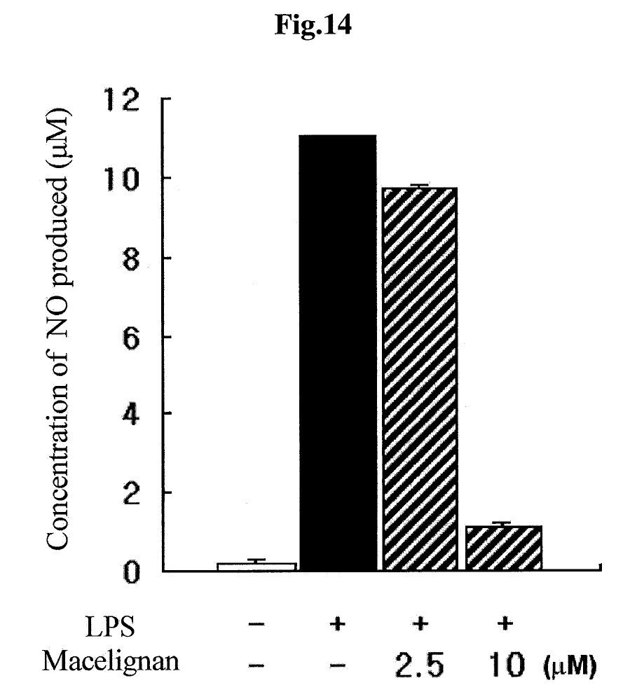

As a result, shown in FIG. 14, the NO production induced by LPS in the tissue-cultured microglia was dose-dependently inhibited by the lignan compound of the present invention. Especially, 10 μM of the lignan compound of the present invention exhibited about 90% inhibition rate.

<6-3> Inhibitory Effect on iNOS and COX-2 Expression

In Example <6-2>, it was observed that the NO production induced by LPS in the tissue-cultured microglia of mouse was inhibited by the lignan compound of the present invention. NO is produced by iNOS enzyme of which expression is induced by activation of microglia. Therefore, the correlation between the reduction of NO production and inhibition of iNOS expression was examined. Additionally, a change in the amount of a protein COX-2 participating in the production of other inflammation-mediating materials was examined. For Western blotting analysis, tissue-cultured microglia was cultured in a 60 mm cell culture dish up to a concentration of 7.5×10⁵ cells/ml. After 1 day of cultivation, the activation of microglia was induced by 1 μg/ml of LPS (Sigma) treatment. Simultaneously, the microglia was also treated with 2.5 and 10 μM of the lignan compound of the present invention together with LPS, and incubated for 16 or 24 hours. The incubated cell was washed with cold PBS 2 times, and then dissolved in a cold lysis buffer (1% SDS, 1 mM Na₃VO₄, 10 mM NaF, 10 mM Tris-Cl, pH 7.4 containing 1× protease inhibitors cocktail). The cell lysate was centrifuged at 4° C. and 12,000×g for 10 minutes and the supernatant was collected. Then, the amount of proteins was quantified according to BCA method. The proteins with the same amount were separated through SDS-PAGE, and transferred into a PVDF membrane. Each membrane was blocked with 3% BSA solution, and washed with TBS-T solution (10 mM Tris-Cl, pH 7.5, 150 mM

NaCl containing 0.1% Tween 20) 3 times. Primary antibodies specific to iNOS (rabbit polyclonal Ab, Upstate, 06-573) and COX-2 (rabbit polyclonal Ab, Santa Cruz Biotechnology, sc-7951) were then added into the membrane and incubated at room temperature for 1 hour. After the membrane was washed with TBS-T solution 3 times, HRP-coupled specific secondary antibodies were added into the membrane, and incubated at room temperature for 1 hour. Again, the membrane was then washed with TBS-T solution 3 times, and each band on the membrane was analyzed using ECL system (Sigma).

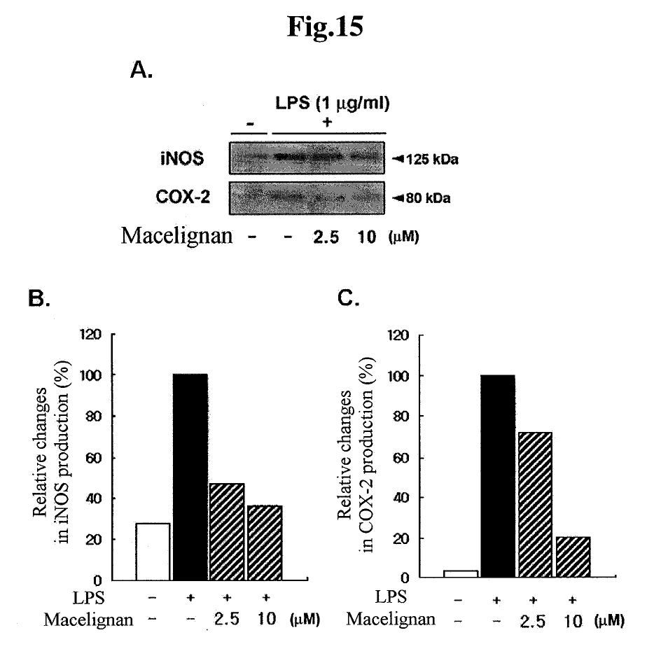

As a result, shown in FIG. 15, the expression of iNOS and COX-2 induced by LPS in the tissue-cultured microglia was inhibited in a concentration dependent manner by the lignan compound of the present invention.

### EXAMPLE 7

Transmission Test of the Lignan Compound of the Present Invention to the Brain

The transmission of the lignan compound of the present invention to the brain was examined.

<7-1> Treatment of Macelignan of the Present Invention and Sample Collection

PE50 tubes are independently inserted into the femoral vein and femoral artery of a 250 g male SD rat, and syringes individually containing physiological saline solution and heparin (25 I.U.) are connected to tubes, respectively. The macelignan isolated and purified in Example 1 was dissolved in DMSO, and intravenously administered into the rat in the amount of 1 mg/kg. 400 μl of the blood samples were collected from the artery at 0.5, 1, 1.5 and 2 minutes after administration. After last sampling, the head of the rat was rapidly cut, and the brain tissue was extracted and slightly washed with physiological saline solution.

<7-2> Sample Treatment

a. Blood Sample Treatment

The blood sample prepared in the Example <7-1> was centrifuged at 3,000 rpm for 5 minutes to obtain 100 μl of blood plasma. 500 μl of ethylacetate was added into the blood plasma and the mixture was agitated with a vortex mixer for 10 minutes. Then, the mixture was centrifuged at 3,000 rpm for 5 minutes to obtain 400 μl of the supernatant. The supernatant was evaporated and dried in the nitrogen stream and reformulated as 100 μl of a mobile phase.

b. Brain Tissue Sample Treatment

The weight of the brain tissue sample prepared in the Example <7-1> was measured, and then saline solution corresponding to the weight of 2 times higher than that of the brain tissue was added into the brain tissue and the mixture was homogenized. The homogenized mixture was agitated with a vortex mixer for 5 minutes, and then the mixture was centrifuged at 3,000 rpm for 5 minutes. 5 ml of ethylacetate was added into 1 ml of the supernatant obtained from the centrifugation and the mixture was agitated with a vortex mixer for 10 minutes. Then, the mixture was centrifuged at 3,000 rpm for 5 minutes to obtain the supernatant. 4 ml of the supernatant was evaporated and dried in the nitrogen stream and reformulated as 100 μl of a mobile phase.

<7-3> Standard Test

1 mg/ml of a stock solution prepared by dissolving macelignan isolated in the Example 1 in methanol was serially diluted. 10 μl of the macelignan solution was added into 90 μl of rat blank blood plasma or 90 μl of rat blank brain homogenate to prepare a desired concentration of plasma sample or brain tissue sample. Then, each sample was treated according to the method of the Example <7-2>. 10 μl of the sample reformulated as 100 μl of the mobile phase was introduced into the LC/MS system (Agilent 1100 Series, Agilent Technologies, Santa Clara, USA). The LC/MS analysis was conducted using 3.0 mm×150 mm C18 Luna column (Phenomenex, Torrance, Calif., USA) under the mobile phase condition of acetonitrile:methanol:deionized distilled water(DDW)=40:40:20.

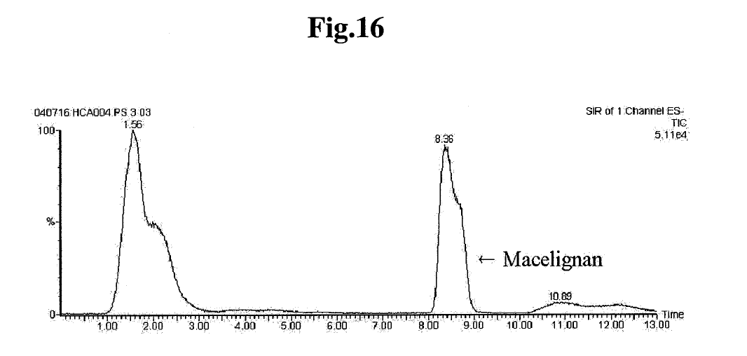

As a result, shown in FIG. 16, macelignan was detected in SIM [327.0-328.0] of ESI negative, and retention time is 8.36 minutes. In the above chromatogram, the area of a macelignan peak was calculated and the linear standard curve regarding the concentration and area of macelignan was prepared.

<7-4> Sample Analysis

The blood sample and brain tissue sample treated in the Example <7-2> were introduced into the LC/MS system, and then the LC/MS analysis was performed according to the method of the Example <7-3> and the area of a macelignan peak was obtained on the chromatograph. The concentration of macelignan was calculated by using the area of the macelignan peak through the standard curve prepared in the Example <7-3>.

<7-5> Transmission Calculation of Macelignan into Brain

In a time-concentration graph of macelignan obtained in the blood sample through the Example <7-4>, AUC₀^{t} (Area Under Curve: AUC) from 0 to the last sample time “tlast”, was obtained with a trapezoidal method (Schaum's Outline of Mathematica, MaGraw-Hill, 2000). Additionally, the amount(Xb) of macelignan was calculated using the concentration of the brain tissue sample. AUC of macelignan may be calculated using the mathematical equation 1.

$${AUC}_{0}^{t} = \frac{1}{2} ( {C}_{0} + {C}_{1} ) \cdot Δ t + \frac{1}{2} ( {C}_{1} + {C}_{2} ) \cdot Δ t + … + \frac{1}{2} ( {C}_{n - 1} + {C}_{n} ) \qquad [ Mathematical Equation 1 ]$$

wherein, C is macelignan concentration, and Δt is a time change.

The brain uptake clearance (CLuptake) value of macelignan transmitting to the brain may be calculated using the mathematical equation 2.

$${CL}_{uptake} = \frac{Xb ( t )}{{AUC}_{0}^{t}} \qquad [ Mathematical Equation 2 ]$$

The mathematical equation 2 was obtained by the following process.

The change of the amount of macelignan in the brain may be represented by the mathematical equation 3.

$$\frac{d {X}_{b}}{d t} = {CL}_{uptake} \cdot {C}_{p} - {CL}_{efflux} \cdot {C}_{b} \qquad [ Mathematical Equation 3 ]$$

wherein CLᵤₚₜₐₖₑ is the brain uptake clearance value of macelignan transmitting to the brain, CL_{efflux} is the brain uptake clearance value of macelignan transmitting to the blood, Cₚ is a concentration of macelignan in the blood, and C_{b} is the concentration of macelignan in the brain tissue.

On the assumption that C_{b} is close to 0 immediately after injection, the mathematical equation 3 may be represented by the mathematical equation 4.

$$\frac{d {X}_{b}}{d t} = {CL}_{uptake} \cdot {C}_{p} \qquad [ Mathematical Equation 4 ]$$

If the both sides are integrated from t=0 to t=t, the mathematical equation 4 may be represented by the mathematical equation 5.

$${\int }_{0}^{t} d {X}_{b} d t = {CL}_{uptake} {\int }_{0}^{t} {C}_{p} d t \qquad [ Mathematical Equation 5 ]$$

The mathematical equation 5 is calculated into the mathematical equation 6.

X_{b}=CLᵤₚₜₐₖₑ·AUC₀^{t} [Mathematical Equation 6]

Therefore, the brain uptake clearance of macelignan transmitting to the brain may be represented by said mathematical equation 2.

The brain uptake clearance of macelignan transmitting to the brain was calculated using the mathematical equation 2. As a result, shown in Table 2, the brain uptake clearance of macelignan transmitting to the brain was 0.203±0.039 mL/min. On the basis of the result, it could be observed that the transmission to the brain of macelignan was relatively satisfactory.

TABLE 2
The brain uptake clearance of macelignan transmitting to the brain

| | Macelignan of the present |
|---|---|
| | invention |
| Xb (ug) | 7.90 ± 1.52 |
| AUC (ug/ml/min) | 38.89 ± 3.62 |
| CLᵤₚₜₐₖₑ (ml/min) | 0.203 ± 0.039 |

### PREPARATION EXAMPLE 1

Preparation of Pharmaceutical Formulations Comprising the Pharmaceutical Composition for Treating or Preventing the Brain Disease According to the Present Invention

<1-1> Preparation of Tablet Formulation

25 mg of the lignan compound or Myristica fragrans extract of the present invention, 26 mg of lactose for direct tableting, 3.5 mg of Avicel (microcrystalline cellulose), 1.5 mg of sodium starch glyconate(disintegration aid) and 8 mg of L-HPC (low-hydroxypropylcellulose; binder) for direct tableting were placed in a U-type mixer and mixed with each other for 20 minutes. After completion of the mixing, 1 mg of magnesium stearate(lubricant) was further added thereto and mixed for 3 minutes. The mixture was subjected to test for quantitative analysis and moisture content analysis, tableted and coated with a film, thus preparing a tablet formulation.

<1-2> Preparation of Syrup Formulation

A syrup comprising 2% (w/v) of the macelignan of the present invention or its pharmaceutically acceptable salt as an active ingredient was prepared in the following manner:

2 g of an acid addition salt of the macelignan of the present invention, 0.8 g of saccharin and 25.4 g of sugar were dissolved in 80 g of hot water. The solution was cooled, and then 8.0 g of glycerin, 0.04 g of fragrance, 4.0 g of ethanol, 0.4 g of sorbic acid and a suitable amount of distilled water were added into the cooled solution. To the mixture, water was added to make a volume of 100 ml.

<1-3> Preparation of Capsule Formulation

50 mg of the lignan compound or Myristica fragrans extract of the present invention, 50 mg of lactose, 46.5 mg of starch, 1 mg of talc and a suitable amount of magnesium stearate were mixed with each other. The mixture was filled in a hard gelatin capsule, thus preparing a capsule formulation.

<1-4> Preparation of Injectable Liquid

An injectable liquid comprising 10 mg of the active ingredient was prepared in the following manner:

1 g of a hydrochloride of the macelignan of the present invention, 0.6 g of sodium chloride and 0.1 g of ascorbic acid were dissolved in distilled water to prepare 100 ml of a solution. The solution was bottled and sterilized by heating it at 20° C. for 30 minutes.

### APPLICATION EXAMPLE 1

Parkinson's Disease

It was known that Parkinson's disease is a central nervous system degenerative brain disease, and shows tremor, muscle rigidity and a loss of physical movement (akinesia) accompanied by mental melancholia. It was also known that the Parkinson's disease is primarily due to the dopaminergenic neuronal cell death of the substantia nigra compacta (Fahn S., Parkinson's disease in: Diseases of the nervous system, (ED) by A. Asbury, G. Mckhann, pp. 1217-1238, Saunders, 1986). It was reported that the neuronal cell death accompanied by the Parkinson's disease is due to oxidative stress, energy metabolism disorder, mutation of mitochondrial genes, excitatory amino acid toxicity, etc. Especially, there are many reports that the neuronal cell death is primarily due to oxidative stress (Fahn S. and Cohen, G., Ann. Neurol. 32(6):804-812, 1992; Foley P. and Riederer P., J. Neurol., 247 [Sppl.2] II/82-II/94, 2000). Accordingly, the pharmaceutical composition of the present invention having protective activity of the brain cells from oxidative stress and inhibitory activity of the brain cell death by apoptosis is highly effective in the treatment or prevention of Parkinson's disease.

### APPLICATION EXAMPLE 1

Alzheimer's Dementia

It was known that Alzheimer's disease is a degenerative neuronal disease accompanied by severe memory disorder and mental illness, and its occurrence rate is about 10-15%/year. According to the autopsy opinion of Alzheimer's disease patients, they show senile plaque and neurofibrillary tangle. The oxidative damage is concerned in occurrence of senile plaque, and the senile plaque itself causes an inflammation response. There was a report on epidemiological evidence that anti-inflammatory agents such as ibuprofen delayed the progress of Alzheimer's disease (McGeer and McGeer, Exp. Gerontol., 33:371-378, 1998). In a mouse of mimic Alzheimer's disease, the treatment of ibuprofen delayed the progress of the disease (Lim et al., J. Neurosci., 20:5709-5714, 2000). Accordingly, the pharmaceutical composition of the present invention having protective activity of the brain cells from oxidative stress and anti-inflammatory activity is highly effective in the treatment or prevention of Alzheimer's disease.

### APPLICATION EXAMPLE 3

Cerebral Apoplexy

Cerebral apoplexy refers to neurological symptoms shown by damage of a corresponding portion of the brain, occurred by clogging or breakage of the blood vessel supplying the blood to the brain. The brain performs many functions. However the damaged portion of the brain does not function, thus exhibiting disorder of physical movement and memory disorder. The cerebral apoplexy is occurred in primarily elderly persons, but can be occurred in persons in the twenties or thirties. The occurrence rate of the cerebral apoplexy is not reduced for 10 years. It was known that the cerebral apoplexy is due to apoptosis induced by over-excitation of a NMDA(N-methyl-D-aspartate) receptor by oversecreted glutamate. The activity of microglia contributes to the NMDA toxicity (Tikka and Koistinaho, J. Immunol., 166(12):7527-33, 2001). Additionally, another reason of the cerebral apoplexy is the oxidative stress. Accordingly, the pharmaceutical composition of the present invention having protective activity of the brain cells from oxidative stress and anti-inflammatory activity is highly effective in the treatment or prevention of cerebral apoplexy.

### APPLICATION EXAMPLE 4

Mild Cognitive Impairment (MCI)

Not a few of old persons show a slight memory disorder, but have no severe difficulty in normal daily life. This situation is referred to as “mild cognitive impairment (MCI)”. The old persons diagnosed as MCI have a high possibility (10-15%/year) to progress into the degenerative neuronal diseases such as Alzheimer's disease within some years. This mild cognitive impairment is a transitional stage between normal senility and initial Alzheimer's disease, and a predromal symptom of degenerative neuronal disease. According to the autopsy opinion of MCI patients, they show senile plaque and neurofibrillary tangle as being similar to Alzheimer's disease. The oxidative damage is concerned in occurrence of senile plaque, and the senile plaque itself causes an inflammation response. Accordingly, the pharmaceutical composition of the present invention having protective activity of the brain cells from oxidative stress and anti-inflammatory activity is highly effective in the treatment or prevention of the mild cognitive impairment.

### CROSS-REFERENCE TO RELATED APPLICATIONS

This application claims priority to Korean Patent Application No. 10-2005-0026963, filed on Mar. 31, 2005, the contents of which are hereby incorporated by reference.

### INDUSTRIAL APPLICABILITY

As described above, the lignan compound of the present invention has the inhibitory effect on various mediators causing the brain cell death and their activity. Especially, it has an enhanced antioxidative effect inhibiting lipid peroxidation and production of reactive oxygen species, an enhanced brain cell-protecting effect inhibiting apoptosis of brain cells, and an enhanced anti-inflammatory effect. Accordingly, the lignan compound of the present invention or Myristica fragrans extract will be highly useful for the treatment or prevention of brain diseases.

1. A pharmaceutical composition for treating or preventing a brain disease, which comprises a lignan compound represented by Formula I or a pharmaceutically acceptable salt thereof as an active ingredient: 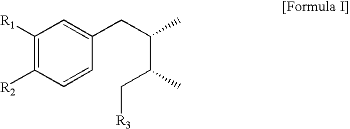
wherein R₁ and R₂ are independently C₁₋₅ alkoxy group or hydroxyl group, and R₃ is or

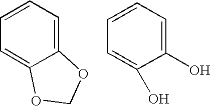

2. The pharmaceutical composition according to claim 1, the lignan compound is macelignan represented by Chemical Formula I:

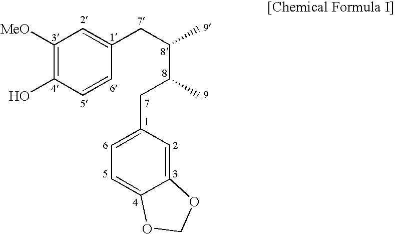

3. A pharmaceutical composition for treating or preventing a brain disease, which comprises water or C₁-C₆ organic solvent extract of Myristica fragrans as an active ingredient.

4. The pharmaceutical composition according to claim 1, wherein the brain disease is any one selected from the group consisting of dementia, Parkinson's disease, cerebral apoplexy, Huntington's disease, Creutzfeldt-Jakob disease, Pick's disease, amyotrophic lateral sclerosis(ALS), Parkinson-ALS-dementia complex, Wilson's disease, progressive supranuclear palsy, mild cognitive impairment and epilepsy.

5. A method for treating or preventing a brain disease, comprising administering to a subject in need thereof an effective amount of the composition according to claim 1.

6. A method for inhibiting a brain cell death, comprising administering to a subject in need thereof an effective amount of a the composition according to claim 1.

7. The method according to claim 5, the lignan compound is macelignan represented by Chemical Formula I:

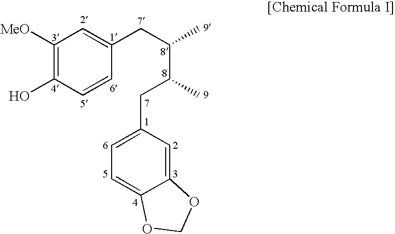

8. A method for treating or preventing a brain disease, comprising administering to a subject in need thereof an effective amount of the composition according to claim 3.

9. A method for inhibiting a brain cell death, comprising administering to a subject in need thereof an effective amount of the composition according to claim 3.

10-14. (canceled)

15. The pharmaceutical composition according to claim 3, wherein the brain disease is any one selected from the group consisting of dementia, Parkinson's disease, cerebral apoplexy, Huntington's disease, Creutzfeldt-Jakob disease, Pick's disease, amyotrophic lateral sclerosis(ALS), Parkinson-ALS-dementia complex, Wilson's disease, progressive supranuclear palsy, mild cognitive impairment and epilepsy.

16. The method according to claim 6, the lignan compound is macelignan represented by Chemical Formula I: [chemical structure]
